(19) United States
(12) Patent Application Publication
(10) Pub. No.: US 2023/0120168 A1
(43) Pub. Date: Apr. 20, 2023

(54) METHOD AND SYSTEM FOR BLOCKCHAIN-BASED MEDICINE-TAKING MANAGEMENT FOR CLINICAL TRIAL SUBJECT

(71) Applicants: LOKKS307 INC., Namdong-gu Incheon (KR); C&R RESEARCH INC., Gangnam-gu Seoul (KR)

(72) Inventors: Byeongyeob OH, Anyang-si Gyeonggi-do (KR); Byungln Yoon, Suwon-si Gyeonggi-do (KR); Jeonil Kang, Gongju-si Chungcheongnam-do (KR)

(73) Assignees: CareSquare Inc., Gangnam-gu, Seoul (KR); C&R RESEARCH INC., Gangnam-gu Seoul (KR)

(21) Appl. No.: 17/755,516

(22) PCT Filed: May 6, 2021
(86) PCT No.: PCT/KR2019/014793

§ 371 Date: Dec. 16, 2022

(30) Foreign Application Priority Data
Oct. 29, 2019 (KR) 10-2019-0135843

(51) Int. Cl. G16H 10/20; G16H 10/60; G16H 20/10

(52) U.S. Cl. CPC G16H 10/20; G16H 10/60; G16H 20/10

(57) ABSTRACT

A method and system for blockchain-based medicine-taking management for a clinical trial subject are disclosed. The method for managing clinical trial medicine-taking comprises the steps of: providing a notification for managing medication for a clinical trial medicine through a medication management app; receiving a medication log of the clinical trial subject by scanning a QR code on the clinical trial medicine through the medication management application; storing the received medication log in a blockchain as medication management information of the clinical trial subject; and verifying the validity of the medication log by using the blockchain.

### TECHNICAL FIELD

The following description relates to a technology for medication management for a clinical trial subject.

### BACKGROUND ART

Generally, in the practice of separation of prescribing and dispensing, in which the physician who provides a medical prescription is independent from the pharmacist who provides the prescription drug (dispenses medication), patients and clinic customers receive prescriptions after consulting physicians in clinics, hospitals, and other medical institutions and are then entitled to get (purchase) the prescribed drugs (medications) from pharmacists.

In this system of separation of prescribing and dispensing, where patients go through a series of steps, from receiving prescriptions from medical institutions to purchasing medications from pharmacists, the patients must present their paper prescription to the pharmacist before their medication can be dispensed.

Physicians and pharmacists should give patients necessary medication instructions (providing information on the names, directions for use and dosage, efficacy and effects, storage methods, side effects, interactions, etc. of the drugs). However, in reality, patients rarely get pertinent medication guidance they need.

As an example of a medication management technology for solving these problems, Korean Laid-Open Patent Publication No. 10-2012-0012130 (published on Feb. 9, 2012), a technology for providing medication management service was disclosed in which pictures of a prescription and medicines are taken through a mobile multimedia device, information on the pictures is sent to a server via a network, and comparison/analysis results (information on the medicines, medication guidance, etc.) are provided via the network.

### DISCLOSURE

### Technical Problem

It is possible to automate clinical trial management and increase the transparency and security of clinical trial data by using an automated system and a block chain.

It is possible to reduce the costs of clinical trials through the automated system and to analyze the effects of medicines in an accurate and verifiable manner based on a blockchain.

### Technical Solution

An exemplary embodiment of the present disclosure provides a clinical trial medication management method executed by a server, the server comprising at least one processor configured to execute computer-readable instructions included in a memory, the clinical trial medication management method comprising: providing, by the at least one processor, a notification for managing medication for a clinical trial medicine through a medication management app installed on a personal terminal of a clinical trial subject; receiving, by the at least one processor, a medication log of the clinical trial subject by scanning a QR code on the clinical trial medicine through the medication management app; and storing, by the at least one processor, the received medication log in a blockchain as medication management information of the clinical trial subject.

According to one aspect, the receiving may comprise receiving medication log data including an image or video of the clinical trial subject taking medication through the medication management app.

According to another aspect, the medication log may be stored in a log storage server linked to the server, and the storing may comprise issuing a ticket for the medication log to be stored in the log storage server, and the storing may comprise storing, in the blockchain, information for checking the integrity of the medication log stored in the log storage server.

According to still another aspect, the storing may comprise a ticket for the medication to be stored in the log storage server, wherein, in the issuing, a ticket containing a signature created with a secret key using a clinical trial identifier, a clinical trial subject identifier, and a medicine identifier, and the log storage server checks the validity of the ticket and stores the data of the medication log based on the validity of the ticket.

According to a further aspect, the personal terminal of the clinical trial subject may create a transaction related to the medication log through the medication management app and sends the transaction to the blockchain, the transaction containing a trial identifier, a clinical trial subject identifier, and a medicine identifier which are values encrypted with a random key, the clinical trial medication management method further comprising checking, by the at least one processor, the validity of the medication log by using at least one of a transaction ID corresponding to given information, the random key, the medication log, and a hash value of the ticket issued for the medication log to be stored.

According to a further aspect, the personal terminal of the clinical trial subject may send the transaction to the blockchain and also send the ID of the transaction, the medication log data, and the random key to the log storage server linked to the server, and the log storage server may send the ID of the transaction, an integrity value of the medication log data, and the random key to the server, the storing comprising: sending the ID of the transaction and the random key to the blockchain; and receiving a value assigned to the random key from the blockchain as the blockchain verifies the transaction received from the personal terminal of the clinical trial subject.

Another exemplary embodiment of the present disclosure provides a server implemented by a computer, comprising at least one processor configured to execute computer-readable instructions included in a memory, wherein the at least one processor provides a notification for managing medication for a clinical trial medicine through a medication management app installed on a personal terminal of a clinical trial subject, receives a medication log of the clinical trial subject by scanning a QR code on the clinical trial medicine through the medication management app, and stores the received medication log in a blockchain as medication management information of the clinical trial subject, according to the transaction created in the medication management app.

### Advantageous Effects

In accordance with embodiments of the present disclosure, it is possible to automate clinical trial management and increase the transparency and security of clinical trial data by using an automated system and a block chain.

In accordance with embodiments of the present disclosure, it is possible to reduce the costs of clinical trials through the automated system and to analyze the effects of medicines in an accurate and verifiable manner based on a blockchain.

### DESCRIPTION OF DRAWINGS

FIG. 1 illustrates an overall medication management system according to an embodiment of the present disclosure.

FIG. 2 is a view illustrating an example of an internal configuration of a computer device according to an embodiment of the present disclosure.

FIG. 3 illustrates a process of keeping a medication log according to an embodiment of the present disclosure.

FIG. 4 illustrates a process of checking the validity of a medication log according to an embodiment of the present disclosure.

FIGS. 5 and 6 illustrate a data processing process for storing medication log data according to an embodiment of the present disclosure.

FIGS. 7 to 9 are exemplary views for explaining a user interface screen of a clinical trial medication management app according to an embodiment of the present disclosure.

FIG. 10 illustrates a medication management automation process according to an embodiment of the present disclosure.

FIG. 11 illustrates an exemplary form of a medication transaction according to an embodiment of the present disclosure.

FIG. 12 illustrates a process for checking the validity of a medication log according to an embodiment of the present disclosure.

FIGS. 13 to 15 illustrate a blockchain with a fixed size and a value verification process using the same according to an embodiment of the present disclosure.

### BEST MODE

Hereinafter, an embodiment of the present disclosure will be described in detail with reference to the accompanying drawings.

Embodiments of the present disclosure relate to a technology for medication management of a clinical trial subject.

Embodiments, including those specifically disclosed herein, may allow a terminal of a clinical trial subject to interact with a clinical trial medication management platform and verify clinical trial data based on a blockchain.

A clinical trial involves fives phases: 1) recruiting subjects to participate in a clinical trial; 2) giving medication instructions/delivering medicines; 3) medication time management by individuals; 4) monitoring medication compliance (regularly, such as by collecting empty bottles)/receiving reports on side effects; and 5) analyzing results. In this case, the management of medication times by individuals may have many problems. For instance, a person subjected to a test (a clinical trial subject) might do problematic behaviors, such as pretending to have taken medicine when they haven't or failing to take medication on time as prescribed. Such behaviors may bring about misleading outcomes regarding the efficacy and side effects of the medication. Thus, it is critical in clinical trials to control the behaviors of subjects as much as possible. This may significantly increase the time and costs for clinical trials. Besides, administrative staff may make mistakes in recording clinical trial data, or the data may be intentionally manipulated for economic gain.

The present embodiments are capable of automating clinical trial medication management such as reminders to take medication and recording side effects before and after taking medication, through a dedicated app installed on a terminal of a clinical trial subject.

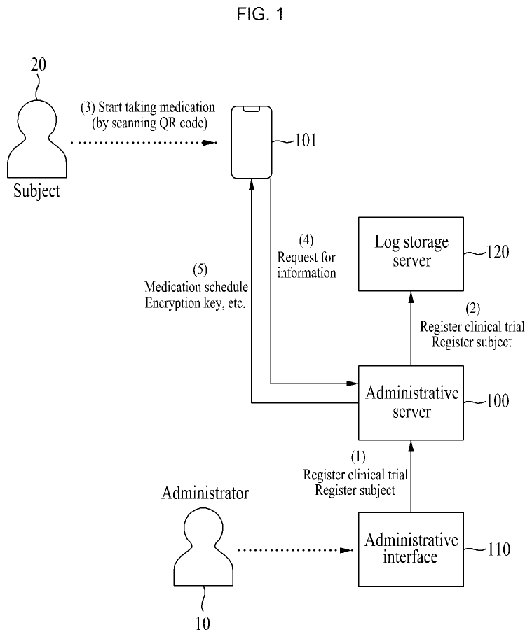

FIG. 1 illustrates an overall medication management system according to an embodiment of the present disclosure.

Referring to FIG. 1, the medication management system according to the present disclosure may include an administrative server 100, an administrative interface 110 for an administrator 10, a log storage server 120, a terminal 101 of a subject 20, and a blockchain network 300.

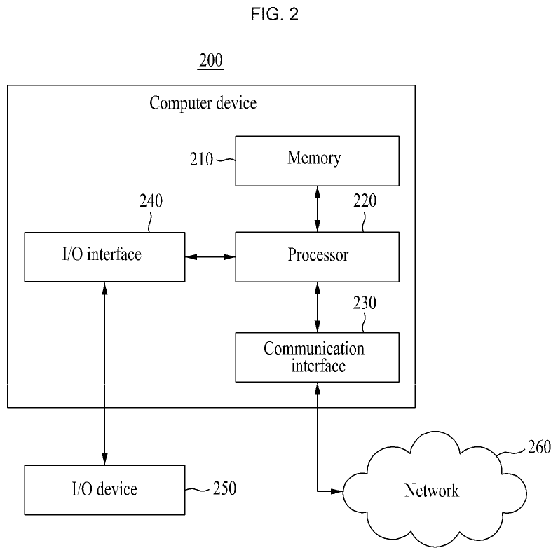

FIG. 2 is a view illustrating an example of an internal configuration of a computer device according to an embodiment of the present disclosure. As described previously, the administrative server 100, the log storage server 120, the terminal 101 of the subject 20, and a terminal of the administrator 10 each may be implemented as physical hardware equipment such as the computer device 200 of FIG. 2, and in some embodiments, may be implemented as a combination of two or more computer devices.

As illustrated in FIG. 2, the computer device 200 may include a memory 210 a processor 220, a communication interface 230, and an input/output (I/O) interface 240. The memory 210 is a computer-readable recording medium, and may include a permanent mass storage device, such as random-access memory (RAM), read only memory (ROM), and a disk drive. In this case, the permanent mass storage device, such as ROM and a disk drive, may be included in the computer device 200 as a permanent storage device separated from the memory 210. Furthermore, an operating system and at least one program code may be stored in the memory 210. Such software elements may be loaded from a computer-readable recording medium different from the memory 210 to the memory 210. Such a separate computer-readable recording medium may include computer-readable recording media, such as a floppy drive, a disk, a tape, a DVD/CD-ROM drive, and a memory card. In another embodiment, the software elements may be loaded onto the memory 210 through the communication interface 230 not through a computer-readable recording medium. For example, the software elements may be loaded onto the memory 210 of the computer device 200 based on a computer program installed by files received over the network 260.

The processor 220 may be configured to process an instruction of a computer program by performing basic arithmetic, logic and I/O operations. The instruction may be provided to the processor 220 by the memory 210 or the communication interface 230. For example, the processor 220 may be configured to execute an instruction received according to a program code stored in a recording device, such as the memory 210.

The communication interface 230 may provide a function for enabling the computer device 200 to communicate with another device (e.g., the above-mentioned storage devices) over the network 260. For example, a request, an instruction, data or a file generated by the processor 220 of the computer device 200 according to a program code stored in a recording device, such as the memory 210, may be forwarded to other devices over the network 260 under the control of the communication interface 230. Inversely, a signal, an instruction, data or a file from another device may be received by the computer device 200 through the communication interface 230 of the computer device 200 over the network 260. The signal, instruction or data received through the communication interface 230 may be forwarded to the processor 220 or the memory 210, and the file received through the communication interface 230 may be stored in a storage medium (the aforementioned permanent storage device which may be further included in the computer device 200.

The I/O interface 240 may be means for interfacing with an input/output (I/O) device 250. For example, the input device may include a device, such as a microphone, a keyboard, or a mouse. The output device may include a device, such as a display, or a speaker. For another example, the I/O interface 240 may be means for interfacing with a device in which functions for input and output have been integrated into one, such as a touch screen. At least one of the I/O devices 250 may be configured as one device along with the computer device 200.

Furthermore, in other embodiments, the computer device 200 may include more or fewer components than those shown in FIG. 2. However, there is no need to clearly illustrate most prior art components. For example, the computer device 200 may be implemented to include at least part of the aforementioned I/O devices 250 or to further include other components such as a transceiver, a database, and the like.

Referring again to FIG. 1, once the administrator 10 registers details of a clinical trial and a subject 20 in the administrative server 100 through the administrative interface 110, the administrative server 100 registers this information in the log storage server 120. Afterwards, the subject 20 may install a dedicated application for clinical trial medication management on a personal terminal (e.g., smartphone) 101 and then start registration of medication-related information through a QR code. The terminal 101 of the subject 20 may access the address of the administrative server 100 included in the QR code and obtain a default medication schedule or an encryption key. In some cases, it may get a ticket issued.

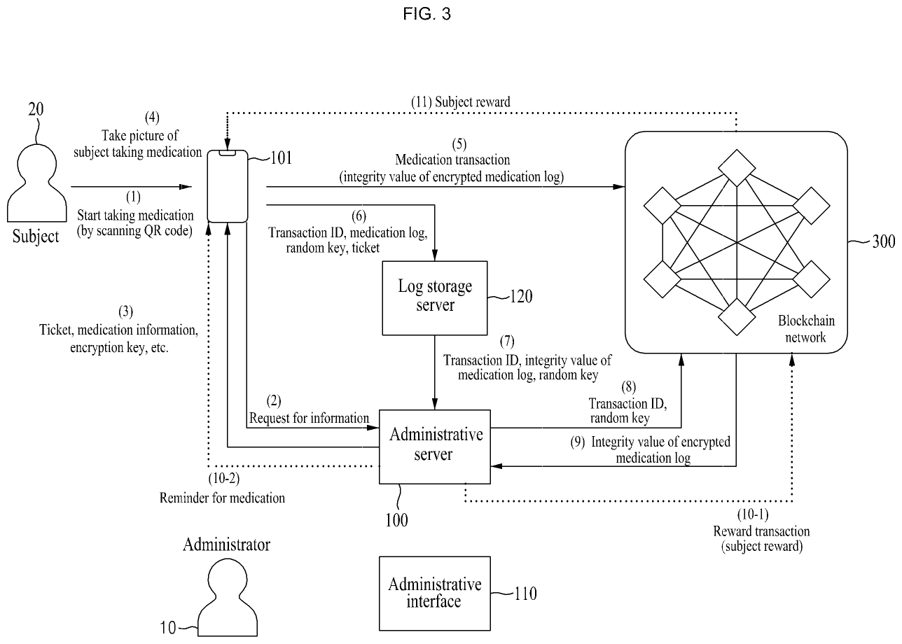

FIG. 3 illustrates a process of keeping a medication log according to an embodiment of the present disclosure.

Referring to FIG. 3, the subject 20 starts taking a picture of medication with the personal terminal 101 (by scanning a QR code on the container or packaging of their medicine). The personal terminal 101 of the subject 20 requests the administrative server 100 for medication information and a ticket, and the administrative server 100 sends the medication information specified for the subject 20 and the ticket (issued) to the personal terminal 101 of the subject 20. The personal terminal 101 of the subject 20 takes a picture of the subject 20 taking medication according to the medication schedule, and then generates a medication transaction and sends it to the blockchain network 300. In addition, the personal terminal 101 of the subject 20 sends a transaction ID, medication log data, a random key, a ticket, etc. to the log storage server 120, and the log storage server 120 sends the transaction ID of the subject 20, an integrity value of the medication log data, the random key, a hash value of the ticket, etc. to the administrative server 100. The administrative server 100 sends an inquiry to the blockchain network 300 about the ID of the medication transaction and the random key. The blockchain network 300 verifies the transaction and sends the administrative server 100 a value assigned to the random key (an integrity value of an encrypted medication log). If the integrity of the medication log is ensured, the administrative server 100 creates a reward transaction and sends it to the blockchain network 300. Then, the reward transaction is sent to the subject 20 through the blockchain network 300. If the subject 20 does not take their medication after a prescribed time, the administrative server 100 pushes reminders for medication to the personal terminal 101 of the subject 20.

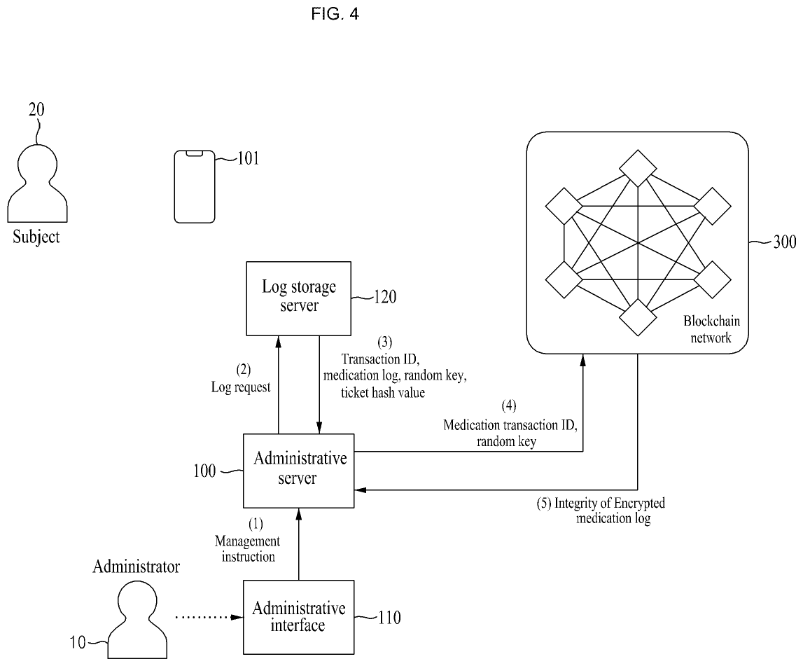

FIG. 4 illustrates a process of checking the validity of a medication log according to an embodiment of the present disclosure.

Referring to FIG. 4, the administrator 10 sends an inquiry to the administrative server 100 about a particular subject 20 or a particular date through the administrative interface 110. The log storage server 120 sends a transaction ID and medication log that match the inquiry. Upon receiving them, the administrative server 100 inquires the blockchain network 300 about the medication transaction ID. The blockchain network 300 sends a verifiable medication transaction. Lastly, the administrative server 100 checks the validity of the medication log.

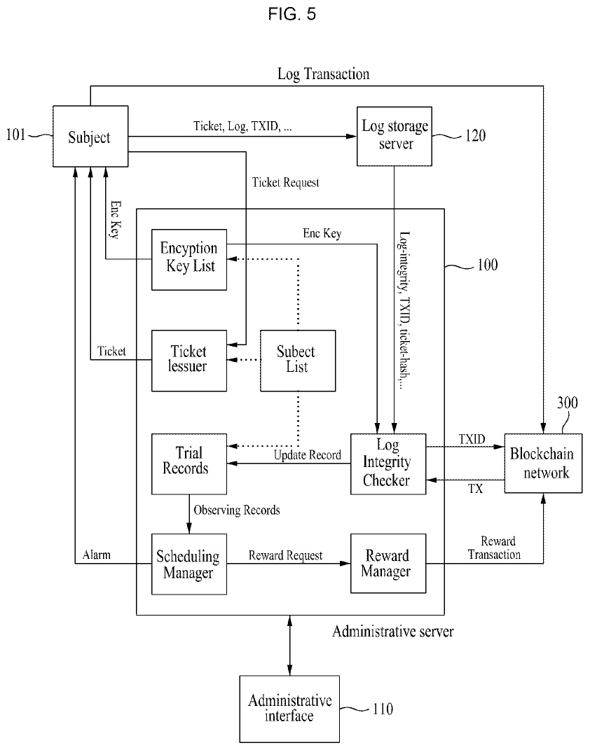

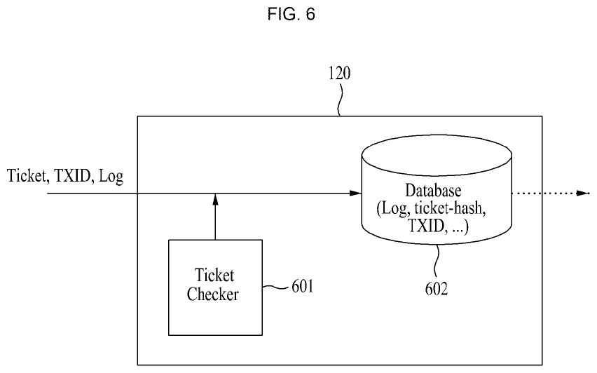

For keeping a medication log, data is processed among the personal terminal 101 of the subject 20, the administrative server 100, the log storage server 120, and the blockchain network 300, as illustrated in FIG. 5. In this case, as illustrated in FIG. 6, the log storage server 120 may receive a ticket (issued), medication log data, a transaction ID (TXID), etc. from the personal terminal 101 of the subject 20, verify them through a ticket checker 601, and store the verified data in a database 602.

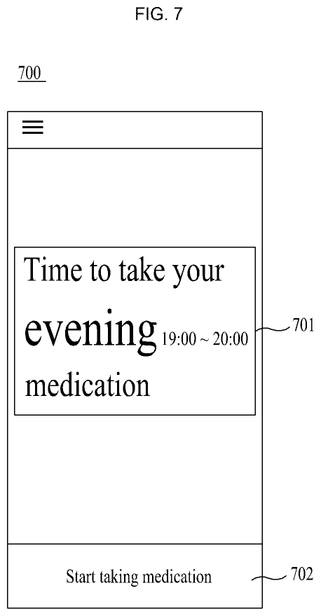

FIGS. 7 to 9 are exemplary views for explaining a user interface screen of a clinical trial medication management app according to an embodiment of the present disclosure.

Functions for the subject 20 may include a medication reminder function, a medication calendar function, a clinical trial schedule check function, a block signature function. The medication reminder function is to provide push notifications in advance to remind the subject 20 when to take medication and give guidance to encourage the subject 20 to take medication at a specified time. The medication calendar function is a function that lets the subject 20 view their medication schedule and records, and the clinical trial schedule check function is a function that lets the subject 20 see their scheduled appointments and treatments related to the clinical trial. The block signature function allows the subject 20 to participate as a signer in block storage activities on the blockchain.

Medication log-related functions for the subject 20 may include a medication log keeping function, a medication picture-taking function, and a self-diagnosis function. The medication log keeping function is a function that lets the subject 20 make a record of themselves taking their medicine by scanning a QR code printed on the packaging of the medicine. The medication picture-taking function is a function that lets the subject 20 take a picture of themselves taking medicine. The self-diagnosis function is a function that allows the subject 20 to self-diagnose the efficacy and side effects of the medication and enter them.

Referring to FIG. 7, the personal terminal 101 of the subject 20 may receive a medication reminder 701 set by the administrator 10 through the clinical trial medication management app and display it on a screen of the personal terminal 101. A clinical trial medication management app screen 700 may include a “Take Medication” UI 702 for medication reporting. When it is time for medication as set by the administrator 10, a medication reminder 701 may arrive at the clinical trial medication management app, or when the subject 20 executes the clinical trial medication management app at a certain time, the time for medication set by the administrator 10 may be displayed. When it is time for medication as set by the administrator 10, the “Take Medication” UI 702 may be enabled and displayed on the clinical trial medication management app screen 700.

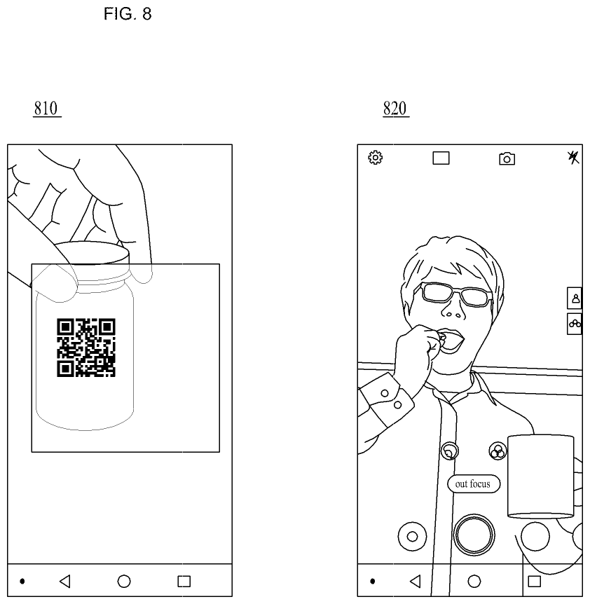

If the subject 20 chooses the “Take Medication” UI 702 from the clinical trial medication management app screen 700, the personal terminal 101 may provide at least one of a QR code scan screen 810 for scanning the QR code of the medicine they are about to take and a video shooting screen 820 for shooting a video of themselves taking the medication, or may provide the QR code scan screen 810 and the video shooting screen 820 sequentially, as illustrated in FIG. 8.

The personal terminal 101 may record medication management information, which the subject 20 enters using the medication log keeping function, in the blockchain 300 through the clinical trial medication management app, in order to send a medication report of the subject 20.

For example, the clinical trial medication management app may send text or voice guidance messages about medication in a step-by-step fashion. If the subject 20 chooses the “Take Medication” UI 702, the clinical trial medication management app may present the QR code scan screen 810 and scan the QR code on the medicine the subject 20 is about to take through the QR code scan screen 810 to provide a notification of which medicine the subject 20 takes. Subsequently, the clinical trial medication management app may present the video shooting screen 820 when the subject 20 requests the next step, and may automatically send a blockchain transaction and medication log data (e.g., video) to the blockchain 300 and the administrative server 100 once video recording is completed through the video shooting screen 820.

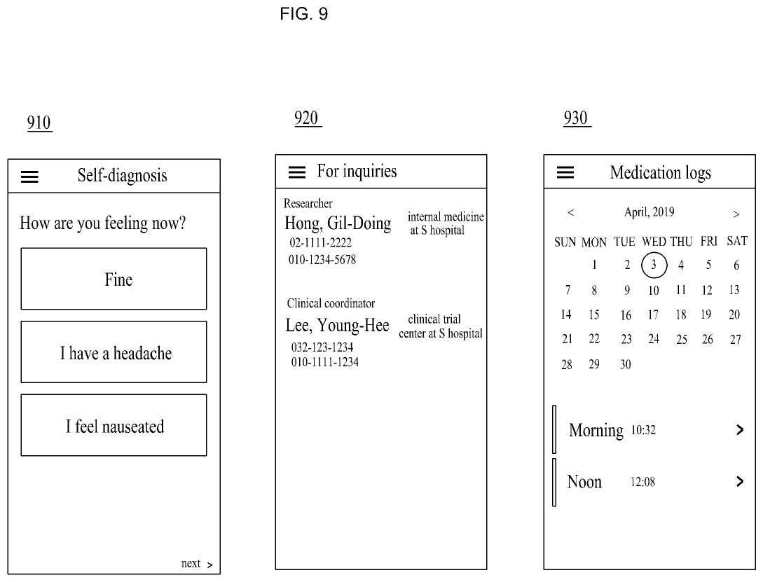

The personal terminal 101 may provide various functions related to medication management according to a request from the subject 20 through the clinical trial medication management app. For example, as illustrated in FIG. 9, the personal terminal 101 may provide a self-diagnosis screen 910 for allowing the subject 20 to self-diagnose the efficacy and side effects of the medication, an inquiry screen 920 for providing contact information of the administrator 10 or clinical trial researchers or allowing the subject 20 to have a chat or call, so that they can make an inquiry about the clinical trial, and a calendar screen 930 for letting the subject 20 view their medication schedule and records.

If the subject 20 chooses a specific date from the calendar screen 930, the personal terminal 101 may provide a detailed screen containing specific medication management information (medication schedule or records) for this date, and, as for the medication schedule, may provide information on the medicine, a medication guide, and so on.

Also, the administrative server 100 may provide the administrator 10 a medication management web page for administrator through the administrative interface 110. The medication management web page for administrator is configured as a web screen including a function for looking up basic information on the clinical trial, a function for entering the profile of the subject, treatment/prescription records, test results, and treatment effects, a function for sending announcements to the subject and sending and receiving messages to and from the subject, a function for setting and managing medication-related information such as the medication schedule, dosage, etc. of the subject, and a function for entering and looking up the progress of the clinical trial of the medication and the outcomes of the trial.

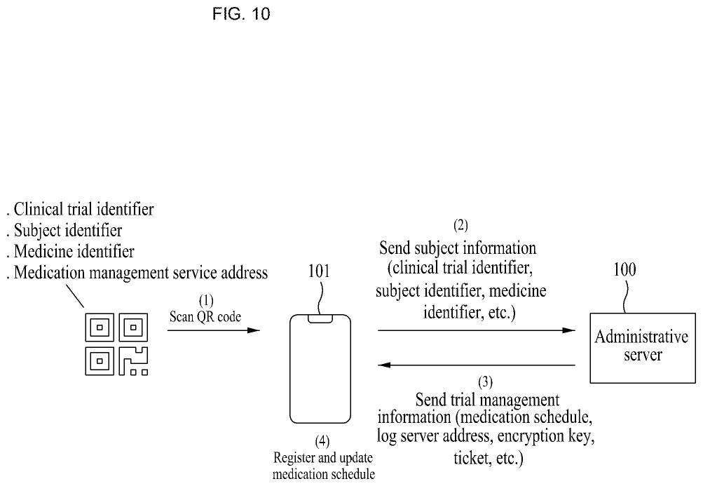

FIG. 10 illustrates a medication management automation process according to an embodiment of the present disclosure.

A medication log contains information about at what time, how, and which user took which medicine. It might be quite difficult for the subject to enter and remember every piece of such data, so automation using the personal terminal 101 such as a smartphone is required.

Referring to FIG. 10, a QR code containing a clinical trial identifier, a subject identifier, the type of medicine, and a medication management service address is printed and attached onto the container or packaging of the medicine, and this QR code is scanned using the terminal 101 of the subject 20. In this case, the medication management service address is information for accessing the administrative server 100, which is used to get medication information online from the administrative server 100. After getting the medication information, the terminal 101 of the subject 20 may automatically register a medication schedule or the like to use it for automatic notifications.

Afterwards, when it is time to take medication, the subject 20 gets notified that their medication procedure is going to start now and then starts the medication procedure. Once the medication procedure is started, the subject 20 answers a brief questionnaire and then shoots an image or the like. Summary information about this is encrypted using an obtained encryption key (public key) and sent to the blockchain 300, and entire log information, along with a ticket, is sent to the log storage server 120.

The encryption key may be different or the same each time. This depends on the policies of the administrative server 100 (medication platform).

A medication log may include text data containing various identifiers and image or video data of the subject 20 taking medication. The blockchain 300 is not suitable for directly storing data that takes up a lot of storage space, such as image or video data, due to its storage space problems. Accordingly, the image or video data is sent separately to the log storage server 120 which is provided by the administrative server 100. In this case, information for checking the integrity of image or video is recorded on the blockchain 300.

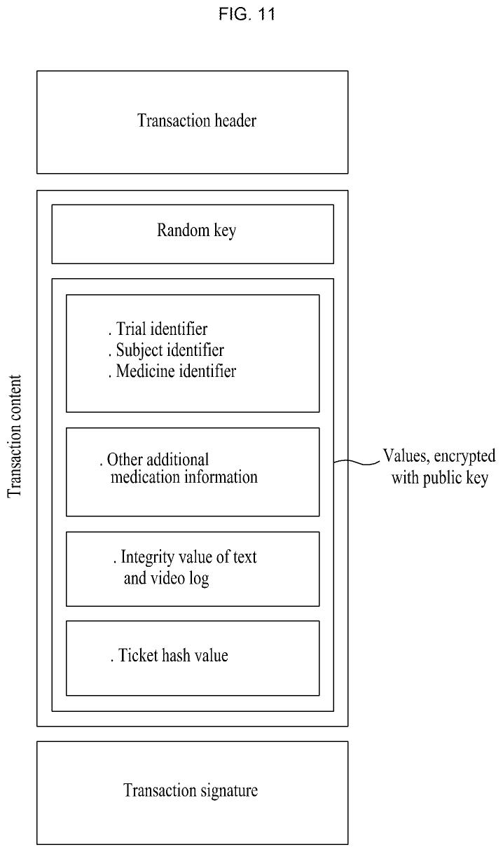

Referring to FIG. 11, a medication transaction sent to the blockchain 300 may come in the following form, and a specific form of the transaction may vary with the blockchain. The content of the transaction may include values encrypted with a random key and a public key that are designed to avoid collisions by using a trial identifier, a subject identifier, a medicine identifier, a time, etc. The values may include the trial identifier, the subject identifier, the medicine identifier, other additional information related to medication, an integrity value, a ticket hash value, and so on.

After the transaction is recorded in the blockchain 300, the transaction ID and the random key are sent to log storage server 120, along with the entire log information.

In order to create a signature for the transaction and record the transaction in the blockchain 300, the subject 20 generally requires as much cryptocurrency as needed for transaction fees. In this case, the administrative server 100 may check the fees paid by the subject 20 and then send as much cryptocurrency as the subject 20 paid for the fees. If there is smart contract functionality that allows a creator of a contract to pay the money, the subject 20 does not have to pay for the fees and also the platform does not have to return the fees.

The administrative server 100 of the medication platform makes sure that the medication log and related transaction sent to the log storage server 120 is properly processed in the blockchain, and pays a reward partially or wholly by cryptocurrency. If the integrity value included in the transaction does not match the log data sent to the log storage server 120, the reward may not be given. The reward may be given immediately after each specified medication time or at regular intervals. The reward may be given simply by paying a certain amount of money, or the reward may be provided as an incentive when the subject 20 has taken medication correctly on time. Such an incentive may encourage or motivate the subject 20 to engage themselves more actively in the clinical trial.

Also, a ticket is required to keep a clinical record in the log storage server 120. The ticket is issued by the administrative server 100, and validated by the log storage server 120. A log with no valid ticket is not stored in the log storage server 120. The subject 20 may access the administrative server 100 through the QR code and get a ticket issued for log storage.

The ticket includes the trial identifier which requests issuance of the ticket, the subject identifier, the medicine identifier, the current time, a user IP address, and a signature created with a secret key of the administrative server 100 based on this information. The log storage server 120 checks the validity of the ticket using other various values other than the log data received from the subject 20. If the ticket is valid, the log storage server 120 stores the ID of the medication transaction, the random key, the medication log, etc.

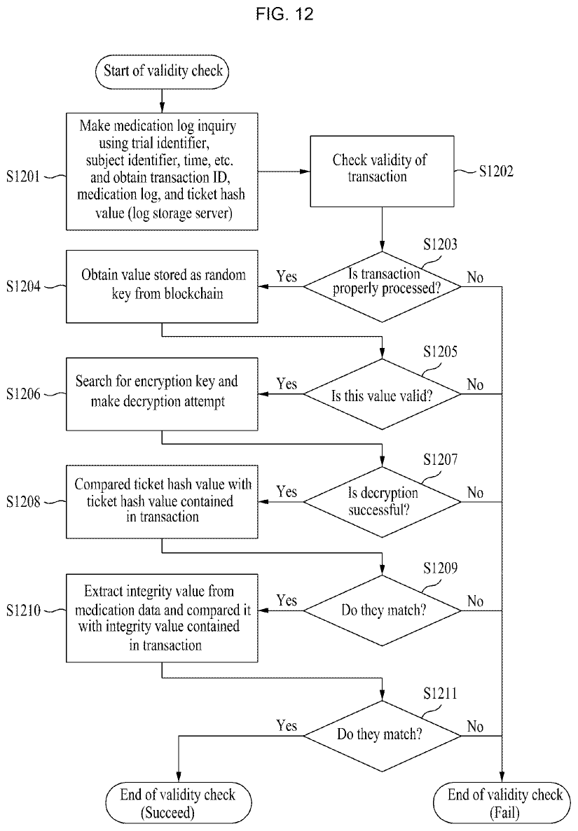

FIG. 12 illustrates a process of checking the validity of a medication log according to an embodiment of the present disclosure.

Referring to FIG. 12, when the log storage server 120 is provided with information such as a trial identifier, a subject identifier, and a time, it returns a medication transaction ID, a random key, a ticket hash value, a medication log, etc. (S1201).

To check the validity of the medication log, the medication transaction generated by the subject 20 is verified (S1202). This function is provided by the blockchain 300 and performed in accordance with a set of procedures of the blockchain.

If the medication transaction generated by the subject 20 is not verified, the medication log is considered invalid (S1203). If the medication transaction generated by the subject 20 is verified, a value stored as the random key is acquired from the blockchain 300, and the validity of the stored value is checked to see whether the value is correct (S1204). For a blockchain having an internal structure described in the present disclosure, the validity of the value is checked in accordance with a method for this type of blockchain.

If the value stored as the random key is not valid, the medication log is considered invalid (S1205). If the value stored as the random key is valid, an attempt is made to decrypt the value with a correct decryption key that can be found by an encryption key manager of the administrative server 100, based on the information such as the trial identifier, the subject identifier and the time (S1206).

If the decryption fails, the medication log is considered invalid (S1207). If the decryption is successful, it is checked whether the ticket hash value included in the transaction matches a ticket hash value sent by the log storage server 120 (S1208).

If the ticket hash value sent by the log storage server 120 does not match the ticket hash value included in the transaction, the medication log is considered invalid (S1209). If the ticket hash values match, an integrity value is extracted from the medication log, and it is checked whether the integrity value matches an integrity value included in the transaction (S1210).

If the integrity value extracted from the medication log does not match the integrity value included in the transaction, the medication log is considered invalid (S1211). If the integrity values match, this means that the medication log has passed all the steps of the validity checking procedure, and the medication log is considered valid.

Although the administrative server 100 of the medication management platform may receive and process transaction content by proxy to avoid the problem of payment of transaction fees, there is a possibility that the platform might issue a transaction only for certain transaction data or manipulate transaction data before sending the transaction to the blockchain 300. Thus, medication transactions are only created by the terminal 101 of the subject 20.

In many blockchains, transactions are divided into transactions for sending currency and transactions for running smart contracts. By this division, medication transactions may fall into the category of transactions for running smart contracts.

However, a large number of blockchains (e.g., Ethereum) does not directly open the source code of smart contracts, but the source code is converted into OP code and stored. These smart contracts are not designed with direct use by general users in mind, but rather with the assumption that only the creators of the contracts (in this case, the medication management platform provider) may use them. It is hard to know precisely what operations the smart contracts perform.

Thus, the medication management platform hardly guarantees the reliability of medication logs if it is built with a typical blockchain in which smart contracts are written in a procedural programming language. This is because a smart contract created with an intention may lead to selective processing of a particular transaction. Accordingly, a blockchain that provides an API for storing key-value pairs or an equivalent function thereof is needed, and transactions for this blockchain need to be accepted.

Lastly, in the blockchain for the medication management platform, key-value pairs are stored as states of the blockchain.

A typical blockchain uses a state tree to verify states, in which the route and position of a node are determined using a key, a value is recorded in the node, and the state root of the node is calculated using a hash tree (e.g., Merkle tree, Merkle Patricia tree, etc.). If the value is changed, the state root is re-calculated along the route of the state tree. If state roots included in a block match, it means that the values of the underlying nodes all match.

The problem is the capacity of the state tree. That is, when there are 1 billion of nodes, it means that, by simple arithmetic, 32 to 64 GB of memory space is additionally required except for state values. For this reason, many blockchains are designed to do the processing on a disk level. In reality, more requests for state verification may lead to excessive use of disks.

The state tree has as high an error rate in value verification as the probability of hash collision. The blockchain needs to know all the headers of previous blocks in order to rely on a state root value at a specific time. That is, it is not a single block alone that is needed to rely on a specific value at a specific time. If there are no block headers, the only method available is to request the values of a plurality of block chain nodes.

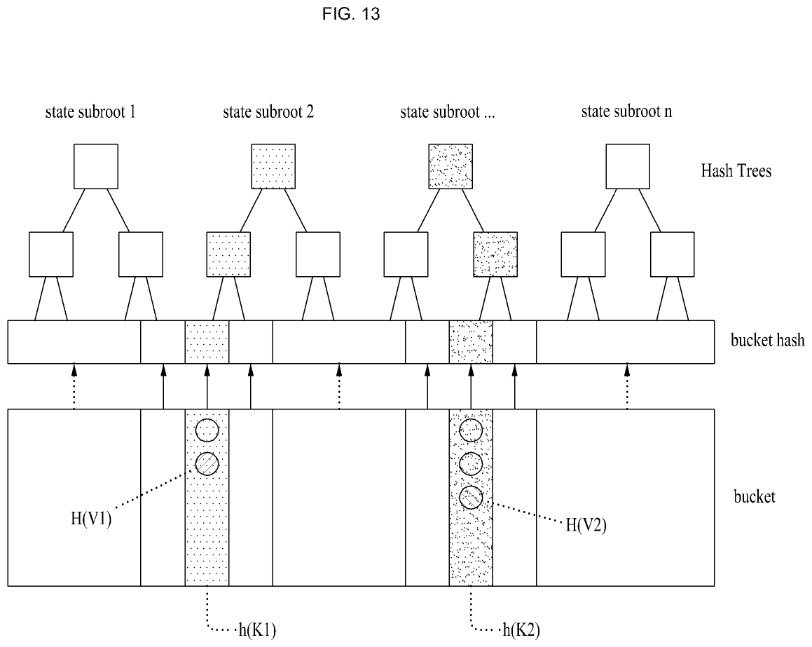

A blockchain with a fixed size and a value verification method using the same are as shown in FIG. 13.

A bucket set B={B_0, B_1, . . . . B_{q−1}}, which is made up of q buckets each containing a maximum of m fingerprints, and multiple hash trees are used.

A structure for verifying key-value pairs (K,V) is added or deleted by employing part of a d-left hashing algorithm. One key-value pair (K,V) has a fingerprint fᵥ, which may be calculated by a cryptographic hash function as in Equation 1.

fᵥ=H₁^{S}(V) (Equation 11

wherein s is the size of the fingerprint, which is around 16 to 32.

The key-value pair (K,V) is converted into a value p₁, p₂, . . . , p_{d} between b and (e-1) by d independent hash functions h₁, h₂, . . . , h_{d} (Equation 2) (b<=p₁, p₂, . . . , p_{d}<=e−1, b>=0, e<q). The hash functions may vary with the key K.

pᵢ=hᵢ(K)mod q [Equation 2]

Using these values, the bucket with the fewest fingerprints is selected from the bucket set B (Equation 3).

p=argminₚ_{₁}_{∈(p}_{₁}_{,p}_{₂}_{, . . . ,p}_{_{d}}₎(|Bₚ_{₁}|) [Equation 3]

If there are multiple bucket positions with the fewest fingerprints, the bucket with the lowest positional value is selected. The fingerprint fᵥ of the key-value pair (K,V) is added to the selected bucket.

For deletion, the value p₁, p₂, . . . , p_{d} is calculated in the same manner as above, the corresponding buckets are checked for the presence of the fingerprint fᵥ, and the fingerprint is deleted from the bucket where it is present.

Once a fingerprint is added to or deleted from a bucket, the hash value of the bucket is re-calculated.

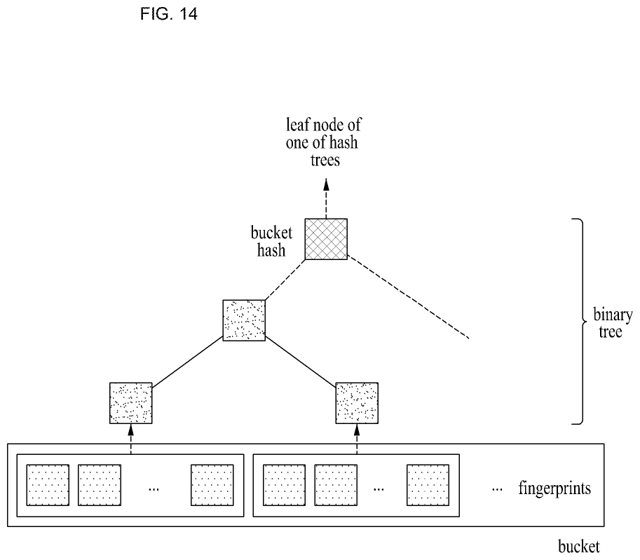

A simple way of obtaining the hash value of the bucket is to hash all the fingerprints included in the bucket. If the maximum number m of fingerprints in the bucket is equal to or greater than a certain value, a hash tree having the construction illustrated in FIG. 14 may be additionally used to calculate the hash value of the bucket.

Referring to FIG. 14, a set of hash trees is made up of n complete binary hash trees in which each leaf node contains a bucket hash value. That is, the number q of buckets is multiples of powers of two. As a consequence, n state subroots exist at a point in time on the blockchain.

In a case where a transaction is included in a certain block and key-value pairs are changed as a result of processing the transaction, the values of a plurality of state subroots are changed depending on the bucket affected by the change. The block header only contains the values of state subroots that are changed in previous blocks. It does not contain the values of state subroots that are not changed.

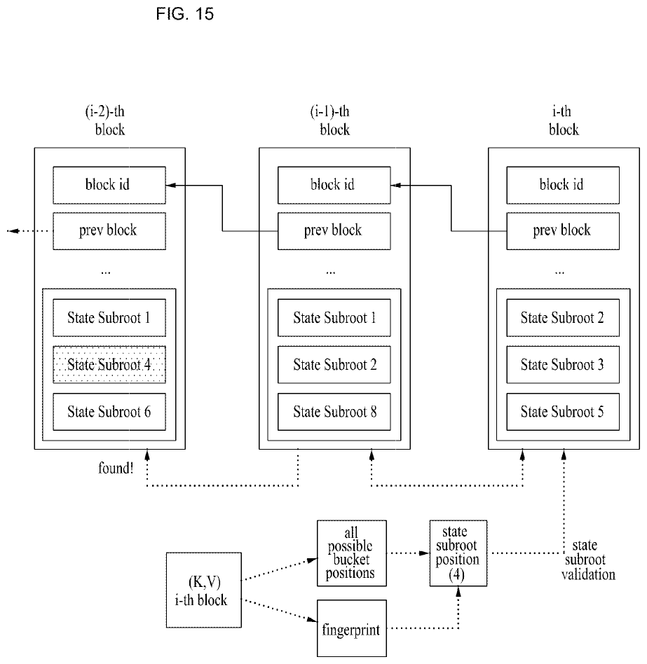

As illustrated in FIG. 15, if a certain user requests the value V of a key K in a particular block, a blockchain node sends 1) the hash value of the bucket to which the key K belongs, 2) a proof for verifying the bucket hash value (the bucket itself or siblings of a fingerprint hash tree), and 3) siblings of the hash tree to which the bucket belongs.

Upon receiving them, the user 1) confirms that the value V has been properly used to create the bucket hash value, 2) calculates a state subroot by using the bucket hash value and the siblings of the hash tree, and 3) checks whether the calculated state subroot matches any of all possible state subroots created from the key K. A particular state subroot of an i-th block is the last state subroot of a block preceding the i-th block. For example, in FIG. 15, the value of a fourth state subroot of the 1-th block is recoded in the (i−2)-th block.

In hot and cold bucket methods, only some buckets may be selected depending on the key. For example, only buckets in a specific range may be used when setting the positions of buckets by using a particular string at the prefix of a key. By doing so, data with a common objective may be gathered in particular buckets.

Limiting the range of buckets depending on the key offers many advantages in administrating the blockchain if a specific application program is used intensively during a certain period of time. Inversely, particular buckets may be moved from a memory area to a disk area and used by intentionally lowering the usability of these buckets. Suppose that the buckets present in memory are referred to as hot buckets and the buckets present in disks are referred to as cold buckets, then the cold buckets and the hot buckets may be adjusted to appropriate levels so that they use less memory than the size of the buckets actually operated by the nodes running the blockchain.

A new structure according to the present embodiment may be designed not to be infinitely extendable, but to have a particular size (e.g., 4 GB) that fits the memory area of a general computer. In the worst case, it might be impossible to add a new key-value pair due to a limitation of the d-left hashing algorithm. In this case, the key may be changed to make another entry attempt. If the limited capacity of 4 GB is all used up so that values cannot be stored any longer, this is beyond the capability of blockchain operation. Thus, additional measures need to be taken, including increasing the maximum size of buckets or increasing the number of buckets.

While the probability of counterfeiting of values in state trees of existing blockchains ranges from ½¹⁶⁰ to ½²⁵⁶, the probability of counterfeiting in this structure may range from ½¹⁶ to ½³² depending on the size of fingerprints. These figures may seem quite low from a cryptographical perspective. Rather, it can be said that a lot of storage space is additionally used to achieve an extremely low probability of counterfeiting because the features of the blockchain allow for a means for verifying values from a plurality of nodes. Moreover, transmission of invalid values in a blockchain where a real-name authentication attempt is made may impose a large burden on the nodes.

In addition, in state trees of existing blockchains in which a single state root value is calculated and contained in a block header, only one changed value may change the state root value. To check every block to see which key the value is changed for, every block needs to be newly verified even if the value is not changed. In this embodiment, although there is a slight increase in values contained in the block header (which is small compared to transactions or the like contained in the block) because of state subroots, this is effective because the key-value needs to be newly verified only in the block in which the state subroots containing the key are changed, by checking which state subroots are contained in the block header. Also, the height of hash tree is decreased due to the state subroots, thereby reducing the number of siblings required for key-value verification.

As seen from above, according to the embodiments of the present disclosure, it is possible to automate clinical trial management and increase the transparency and security of clinical trial data by using an automated system and a block chain. Furthermore, it is possible to reduce the costs of clinical trials through the automated system and to analyze the effects of medicines in an accurate and verifiable manner based on a blockchain.

The aforementioned system may be implemented in the form of a hardware component, a software component, and/or a combination of a hardware component and a software component. For example, the system and components described in the embodiments may be implemented using one or more general-purpose computers or special-purpose computers, such as a processor, a controller, an arithmetic logic unit (ALU), a digital signal processor, a microcomputer, a field programmable gate array (FPGA), a programmable logic unit (PLU), a microprocessor, or any other device capable of executing or responding to an instruction. A processing device may run an operating system (OS) and one or more software applications executed on the OS. Furthermore, the processing device may access, store, manipulate, process, and generate data in response to the execution of software. For convenience of understanding, one processing device has been illustrated as being used, but a person having ordinary skill in the art may understand that the processing device may include a plurality of processing elements and/or a plurality of types of processing elements. For example, the processing device may include a plurality of processors or a single processor and a single controller. Furthermore, a different processing configuration, such as a parallel processor, is also possible.

Software may include a computer program, code, an instruction, or a combination of one or more of these and may configure a processing device so that it operates as desired or may instruct the processing device independently or collectively. The software and/or data may be embodied in a machine, component, physical device, virtual equipment, computer storage medium or device of any type in order to be interpreted by the processing device or to provide an instruction or data to the processing device. The software may be distributed to computer systems connected over a network and may be stored or executed in a distributed manner. The software and data may be stored in one or more computer-readable recording media.

The method according to the embodiment may be implemented in the form of a program instruction executable by various computer means and stored in a computer-readable recording medium. The medium may continuously store a computer-executable program or may temporarily store the program for execution or download. Furthermore, the medium may be various recording means or storage means in the form of a single piece of hardware or a combination of several pieces of hardware. The medium is not limited to a medium directly connected to a computer system, but may be one distributed over a network. Examples of the medium include magnetic media such as a hard disk, a floppy disk and a magnetic tape, an optical recording medium such as CD-ROM and DVD, a magneto-optical medium such as a floptical disk, ROM, RAM, and flash memory, which are configured to store and execute program instructions. Also, other examples of the medium may include recording media and storage media managed by application stores distributing applications or by websites, servers, and the like supplying or distributing other various types of software

### MODE FOR DISCLOSURE

As described above, although the embodiments have been described in connection with the limited embodiments and the drawings, those skilled in the art may modify and change the embodiments in various ways from the description. For example, the relevant results may be achieved even when the described technologies are performed in a different order than the described methods, and/or even when the described components such as systems, structures, devices, and circuits are coupled or combined in a different form than the described methods or are replaced or substituted by other components or equivalents.

Therefore, other implementations, other embodiments, and equivalents to the claims are also within the scope of the following claims.

1. A clinical trial medication management method executed by a server, the server comprising at least one processor configured to execute computer-readable instructions included in a memory,
the clinical trial medication management method comprising:
providing, by the at least one processor, a notification for managing medication for a clinical trial medicine through a medication management app installed on a personal terminal of a clinical trial subject;
receiving, by the at least one processor, a medication log of the clinical trial subject by scanning a QR code on the clinical trial medicine through the medication management app; and
storing, by the at least one processor, the received medication log in a blockchain as medication management information of the clinical trial subject.

2. The clinical trial medication management method of claim 1, wherein the receiving comprises receiving medication log data including an image or video of the clinical trial subject taking medication through the medication management app.

3. The clinical trial medication management method of claim 1, wherein the medication log is stored in a log storage server linked to the server, and the storing comprises storing, in the blockchain, information for checking the integrity of the medication log stored in the log storage server.

4. The clinical trial medication management method of claim 3, wherein the storing comprises issuing a ticket for the medication log to be stored in the log storage server,
wherein, in the issuing, a ticket containing a signature created with a secret key using a clinical trial identifier, a clinical trial subject identifier, and a medicine identifier, and the log storage server checks the validity of the ticket and stores the data of the medication log based on the validity of the ticket.

5. The clinical trial medication management method of claim 1, wherein the personal terminal of the clinical trial subject creates a transaction related to the medication log through the medication management app and sends the transaction to the blockchain,
the transaction containing a trial identifier, a clinical trial subject identifier, and a medicine identifier which are values encrypted with a random key,
the clinical trial medication management method further comprising checking, by the at least one processor, the validity of the medication log by using at least one of a transaction ID corresponding to given information, the random key, the medication log, and a hash value of the ticket issued for the medication log to be stored.

6. The clinical trial medication management method of claim 5, wherein the personal terminal of the clinical trial subject sends the transaction to the blockchain and also sends the ID of the transaction, the medication log data, and the random key to the log storage server linked to the server, and the log storage server sends the ID of the transaction, an integrity value of the medication log data, and the random key to the server,
the storing comprising:
sending the ID of the transaction and the random key to the blockchain; and
receiving a value assigned to the random key from the blockchain as the blockchain verifies the transaction received from the personal terminal of the clinical trial subject.

7. A server implemented by a computer, comprising at least one processor configured to execute computer-readable instructions included in a memory,
wherein the at least one processor provides a notification for managing medication for a clinical trial medicine through a medication management app installed on a personal terminal of a clinical trial subject, receives a medication log of the clinical trial subject by scanning a QR code on the clinical trial medicine through the medication management app, and stores the received medication log in a blockchain as medication management information of the clinical trial subject, according to the transaction created in the medication management app.
